SEQUENCE LISTING (12 sequences)

SEQ ID NO: 1 - DNA, 21 bp, Artificial Sequence
  Synthesized oligonucleotide
agaccgtaattgtggctgcac

SEQ ID NO: 2 - DNA, 21 bp, Artificial Sequence
  Synthesized oligonucleotide
gggaagccgcctataccaaga

SEQ ID NO: 3 - DNA, 21 bp, Artificial Sequence
  Synthesized oligonucleotide
tgtcggctctgctacttcaag

SEQ ID NO: 4 - DNA, 21 bp, Artificial Sequence
  Synthesized oligonucleotide
cgcgactactacaactttcca

SEQ ID NO: 5 - DNA, 21 bp, Artificial Sequence
  Synthesized oligonucleotide
cctgctaatcaagtcacacat

SEQ ID NO: 6 - DNA, 21 bp, Artificial Sequence
  Synthesized oligonucleotide
ccttcagactttgcttcccat

SEQ ID NO: 7 - DNA, 31 bp, Artificial Sequence
  Synthesized primer
tcataggcatcgacgcggggcagtttttccc

SEQ ID NO: 8 - DNA, 31 bp, Artificial Sequence
  Synthesized primer
gggaaaaactgccccgcgtcgatgcctatga

SEQ ID NO: 9 - DNA, 31 bp, Artificial Sequence
  Synthesized primer
ggtgccaccatgaaggccttttgcggcacac

SEQ ID NO: 10 - DNA, 31 bp, Artificial Sequence
  Synthesized primer
gtgtgccgcaaaaggccttcatggtggcacc

SEQ ID NO: 11 - DNA, 34 bp, Artificial Sequence
  Synthesized primer
cggtgccaccatgaaggacttttgcggcacacct

SEQ ID NO: 12 - DNA, 34 bp, Artificial Sequence
  Synthesized primer
aggtgtgccgcaaaagtccttcatggtggcaccg

(19) United States
(12) Patent Application Publication
(10) Pub. No.: US 2011/0206689 A1
(43) Pub. Date: Aug. 25, 2011

(54) Molecular Determinants Associated With Prostate Cancer And Methods Of Use Thereof

(75) Inventors: William C. Hahn, Newton, MA (US); Atish Choudury, Cambridge, MA (US); Isil Guney, Istanbul (TR)

(73) Assignee: Dana-Farber Cancer Institute, Inc., Boston, MA (US)

(21) Appl. No.: 13/011,416

(22) Filed: Jan. 21, 2011

Related U.S. Application Data
(60) Provisional application No. 61/297,104, filed on Jan. 21, 2010.

(51) Int. Cl. A61K 39/395; A61K 33/00; A61K 31/7088; C12Q 1/48; A61P 35/00

(52) U.S. Cl. 424/158.1; 424/600; 514/44 R; 514/44 A; 435/15; 204/450

(57) ABSTRACT

The present invention provides methods of treating cancer by inhibiting pserine threonine kinase activity and detecting cancer using biomarkers.

### RELATED APPLICATIONS

This application claims the benefit of U.S. Ser. No. 61/297,104, filed Jan. 21, 2010, the content of which is incorporated herein by reference in its entirety.

### INCORPORATION-BY-REFERENCE OF SEQUENCE LISTING

The contents of the text file named “20363-053001US_ST25.txt”, which was created on Mar. 3, 2011 and is 3 KB in size, are hereby incorporated by reference in their entirety.

### FIELD OF THE INVENTION

The present invention relates generally to the identification of genetic determinents effecting prostate cancer and methods of using such determinents in the screening, prevention, diagnosis, therapy, monitoring, and prognosis of cancer.

### BACKGROUND OF THE INVENTION

Prostate cancer is currently the most common type of cancer in American men and the second leading cause of cancer related death in this population. In its advanced stages, prostate cancer metastasizes preferentially to bone, where it forms osteoblastic lesions. After initial treatment with androgen ablation therapy, most metastatic prostate cancers become hormone-refractory and lethal. The major cause of morbidity and mortality from prostate cancer is advanced stage, androgen independent disease.

There is currently no effective therapy for hormone refractory prostate cancer and there is currently no marker specific for hormone refractory prostate cancer. In addition to an urgent need for new therapies, there is also a need for diagnostic tests of hormone refractory-prostate cancer. Such tests would indicate which patients have hormone refractory cancer cells at diagnosis and where they are located. This information would have a profound impact on initial therapy. In addition, markers of hormone independent prostate cancer could be used to detect recurrent disease and could be used as therapeutic targets.

Despite recent advances in the diagnosis and treatment of localized prostate cancer, little progress has been made in the fight against advanced disease. Virtually all patients treated with hormone ablation therapy will go on to develop androgen independent recurrences, for which there currently is no therapy. Knowledge of the specific molecular events underlying the progression of prostate cancer to androgen independence is limited.

An understanding of the mechanism of androgen independent growth would provide the framework for the development of rational therapies. The development of specific markers of androgen independent growth would lead to the early identification of patients at risk to fail surgical or hormonal therapy and to the selection of patients for alternative therapies.

Accordingly, a need exists for more accurate models of human cancer that can be used together with complex human datasets to identify robust biomarkers that can be used to predict the occurrence and the behavior of cancer, particularly at an early stage.

### SUMMARY OF THE INVENTION

In one aspect the invention provides a method of treating, alleviating a symptom of hormone-refractory prostate cancer or delaying the onset of androgen-independent prostate tumor growth in a subject by administering to a subject a compound that inhibits the expression or activity of a serine threonine kinase. The compound inhibits the expression of a serine threonine kinase nucleic acid or polypeptide. For example the compound inhibits the expression or activity of a thymidine kinase 1 (TK1) a uridine-cytidine kinase 2 (UCK2), a tyrosine kinase non-receptor 2 (TNK2), a S-phase kinase-associated protein 2 (SKP2), a plasminogen activator, urokinase (PLAU) or a hepatocyte growth factor-regulated tyrosine kinase substrate (HGS). Preferably, the compound inhibits a serine threonine kinase polypeptide activity independent of phosphorylation. The compound is for example an organic compound, a small inorganic compound, a nucleic acid, an antisense oligonucleotide, an siRNA, or an antibody.

In another aspect the invention provide a method of assessing the risk of a subject developing a hormone-refractory prostate cancer by identifying an increase in expression or copy number of TK1 in a subject derived sample compared to a control sample. An increase indicates an increased risk of developing hormone-refractory prostate cancer. The control sample normal tissue of the same tissue type as in the subject sample.

Also provide by the invention is a method with a predetermined level of predictability for assessing a risk development of hormone-refractory prostate cancer or a metastatic prostate cancer in a subject by measuring the level of one or more kinases in a sample from the subject, and measuring a clinically significant alteration in the level of the one or more kinases in the sample. Alternatively, the level of the one or more kinases is compared to a reference value. The reference value is for example an index value. An alteration (i.e., increase or decrease) indicates an increased risk developing hormone-refractory prostate cancer or metastatic prostate cancer in the subject.

In a further aspect the invention provides a predetermined level of predictability for assessing the progression of a tumor in a subject by detecting the level of one or more kinases kinase substrate (HGS) in a sample from the subject in a first sample from the subject at a first period of time, detecting the level of one or more kinases in a second sample from the subject at a second period of time and comparing the level of the one or more kinases detected in the first sample to the second sample. Alternatively the level of the kinases detected is compared to a reference value.

In another aspect the invention provides a method with a predetermined level of predictability for selecting a treatment regimen for a subject diagnosed with prostate cancer by detecting the level of one or more kinases optionally detecting the level of one or more kinases and the level detected to a reference value. Alternatively the level of one or more kinases is compared to the level detected in a second sample from the subject at a second period of time.

Optionally, the first sample is taken from the subject prior to being treated for the tumor and the second sample is taken from the subject after being treated for the tumor.

Optionally, the methods includes measuring at least one standard parameters associated with the cancer, such as s Gleason score or PSA. The sample is for example, is a tumor biopsy, blood, or a circulating tumor cell in a biological fluid.

The kinases measured include for example thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS). The level of the kinase is measured for example electrophoretically, immunochemically or by non-invasive imaging.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. In vivo screen and chromosomal amplification analysis to identify kinases that promote androgen independence. a) In vivo forward genetic screen for drivers of hormone independence. Androgen dependent LHSR-AR cells infected with the ORF-kinase library in pooled format were injected subcutaneously into female mice and tumor formation monitored. ORF integrants in the androgen independent tumors are listed. b) The significance (x axis; q-value) of recurrent amplifications in human prostate tumors across the 22 autosomes (y-axis) identified by GISTIC analysis. DNA from 39 tumors were hybridized onto SNP arrays and analyzed by GISTIC for regions of amplification.

FIG. 2. TK1 overexpression synergizes with AKT1 to promote hormone independent prostate cancer. a) Androgen independent tumor formation. LHSR-AR cells were infected with the indicated constructs and injected subcutaneously into castrated mice, except where indicated. M-myristoylated; F-Flag tagged. b) AKT1 and TK1 promote nuclear translocation of AR independent of androgen. H&E and immunhistochemical staining for AR in subcutaneous tumor sections derived from LHSR-AR cells infected with the indicated constructs. Staining of androgen dependent GFP tumor is shown as control. Minimum of 3 tumors were stained per sample and representative images are shown. c) TK1 chromosomal amplification in hormone sensitive and refractory tumors. DNA from 16 hormone sensitive (HS) and 23 hormone refractory (HR) human prostate tumors (from FIG. 1b) were hybridized onto SNP arrays and analyzed for TK1 copy number gain (CNG). The number and percentage of tumors with and without amplification are indicated. d) TK1 protein expression in hormone sensitive and refractory tumors. Prostate tumor microarrays containing 73 hormone sensitive (HS) and 11 hormone resistant (HS) tumor cores were analyzed for TK1 protein expression by immunohistochemistry. The number and percentage of tumors negative and positive for TK1 expression are indicated. d) Immunoblot analysis for TK1 expression in the indicated prostate cancer cell lines. Actin was used to control for loading. e) Effect of TK1 suppression on the proliferation of prostate cancer cells. Cells were infected with three different hairpins targeting TK1 and a control hairpin targeting GFP, and cell numbers determined 5 days post infection. Relative cell number is normalized to the shGFP control.

FIG. 3. TK1 is androgen induced but does not replace AR for survival. c) TK1 expression is androgen induced. Androgen responsive LNCaP cells cultured in hormone depleted medium were treated with 10 nM of the synthetic androgen, R1881, for the indicated times, and TK1 expression was assayed by immunoblot analysis. Actin was used to control for loading. d) TK1 overexpression does not rescue cells from apoptosis induced by AR ablation. Small hairpins targeting AR were introduced into LNCaP cells infected with TK1, or control uninfected LNCaP cells. Cell proliferation was monitored. Relative proliferation is normalized to the shGFP control.

FIG. 4. TK1 interacts with phospho-AKT1 to drive hormone resistance. a) Putative TK1 interacting proteins. TK1 immunocomplexes were isolated from LSHR-AR/FlagTK1 tumors by Flag immunoprecipitation, and associated proteins identified by mass spectrometry. b,c) TK1 promotes elevated pThr308-AKT levels in b) PrECs, and c) CL-1 cells. Immunoblot analyses for p-AKT, total AKT, p-PDK1, and total PDK1 in LHSR-AR and CL-1 cells expressing the indicated constructs. Actin was used to control for loading. d) TK1 interacts with pThr308-AKT. TK1 and AKT immunocomplexes were isolated from a cell line derived from an androgen independent LHSR-AR/FlagTK1 tumor, and immunoblotted for Flag, total AKT and p308-AKT. e) TK1 regulates p308-AKT in vivo. LHSR-AR cells expressing AKT or AKT and TK1 were injected subcutaneously into mice. Following tumor growth, tumors were harvested precastration or two weeks following castration of the mice p308-AKT expression was assayed by immunohistochemistry in at least 3 tumors per condition. Representative images are shown.

### DETAILED DESCRIPTION OF THE INVENTION

The present invention relates to the identification of molecular determinents associated with conferring subjects with hormone refractory prostate cancer. Accordingly, the molecular determinents are useful in identifying individuals who have or are at risk for developing hormone refractory prostate and/or metastatic prostate cancer. In addition, the molecular determinents are useful as therapeutic targets for treating hormone refractory prostate cancer.

Human cancers harbor innumerable genetic and epigenetic alterations presenting formidable challenges in deciphering those changes that drive the malignant process and dictate a given tumor's clinical behavior. The need for accurately predictive biomarkers reflective of a tumor's malignant potential is evident across many cancer types, particularly prostate cancer, where current management algorithms result in either under-treatment with consequent risk of death or exposure to unnecessary morbid treatments.

Tranformed human prostate epithelial cells (PrEC) that express androgen receptor were produced. These transformed PrEC (LHSR-AR) cells are dependent upon androgens for tumor formation and form tumor in non-castrated male mice but not castrated male mice. The complete dependence of LHSR-AR cells upon androgens for tumorigenicity provides a readily manipulated experimental system to study the development of hormone resistance in prostate cancer. Using this system, an in vivo screen using a human kinase open reading frame expression library was developed to identify kinases that permit prostate tumor growth under castrate conditions.

A total of 16 ORF integrants were identified in the tumors formed in the mice by PCR using vector specific primers. Six of the sixtern kinases were found to be in significant regions of copy number gain. The six kinases include: thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS).

Thymidine kinase 1 (TK1) was identified in two tumors in the screen. In tumor 1B, it was the sole integrant identified, whereas in tumor 1A it was identified along with AKT1 and PHKG2. Since AKT1 activation due to PTEN mutations or chromosomal copy deletions are commonly observed in hormone resistant prostate cancers, we hypothesized that while TK1 on its own may have the capacity to promote hormone resistance, it may synergize with AKT1 activation. To test this hypothesis, castrated male mice were injected with LHSR-AR cells infected with TK1 or AKT1 alone, or TK1 and AKT1 in combination. AKT1 on its own was unable to promote androgen independent tumor formation. While TK1 alone yielded one androgen independent tumor out of nine injections, the AKT1/TK1 combination induced androgen independent tumors at a rate greater than TK1 or AKT1 alone. These findings indicate that TK1 and AKT1 synergize to promote androgen independent tumor growth.

To test whether the kinase activity of TK1 is necessary for its capacity to drive androgen independent tumor formation, a kinase dead mutant of TK1 was generated by substituting catalytic glutamic acid at position +98 to alanine (E98A). This mutant construct was introduced into LHSR-AR/AKT cells, and the cells injected subcutaneously into castrated male mice. E98A-TK1 expressing cells were able to promote hormone independent tumor formation as efficiently as wild type TK1 expressing cells, suggesting that TK1 promotes hormone resistance independent of its kinase activity. (E98A-TK1 expressing cells were unable to promote hormone independent tumor formation, suggesting that TK1 promotes hormone resistance through its kinase activity.

Accordingly, the provide method of treating, alleviating a symptom of, or delaying the onset of hormone refractory prostate cancer by administering to a subject a compound that inhibits the expression or activity of a serine threonine kinase. The invention also provides methods for identifying subjects who have hormone refractory and/or metastatic prostate cancer, or who at risk for developing hormone refractory and/or metastatic prostate cancer by the detection of HRPCDETERMINANTS associated with hormone refractory prostate cancer, including those subjects who are asymptomatic for hormone refractory prostate cancer or the metastatic tumor. HRPCDETERMINANTS include thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS).

HRPCDETERMINANTS are also useful for monitoring subjects undergoing treatments and therapies for cancer, and for selecting or modifying therapies and treatments that would be efficacious in subjects having cancer, wherein selection and use of such treatments and therapies slow the progression of the tumor, or substantially delay or prevent its onset, or reduce or prevent the incidence of tumor metastasis.

Definitions

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“HRPCDETERMINANTS in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. HRPCDETERMINANTS can also include mutated proteins or mutated nucleic acids. HRPCDETERMINANTS also encompass non-blood borne factors or non-analyte physiological markers of health status, such as “clinical parameters” defined herein, as well as “traditional laboratory risk factors”, also defined herein. HRPCDETERMINANTS also include any calculated indices created mathematically or combinations of any one or more of the foregoing measurements, including temporal trends and differences. Where available, and unless otherwise described herein, HRPCDETERMINANTS which are gene products are identified based on the official letter abbreviation or gene symbol assigned by the international Human Genome Organization Naming Committee (HGNC) and listed at the date of this filing at the US National Center for Biotechnology Information (NCBI) web site (www.ncbi.nlm.nih.gov/sites/entrez?db=gene), also known as Entrez Gene.

“HRPCDETERMINANT” OR “HRPCDETERMINANTS” encompass one or more of all nucleic acids or polypeptides whose levels are changed in subjects who have hormone refractory prostate cancer and or metastatic prostate canceror are predisposed to developing hormone refractory prostate and or metastatic prostate cancer, or at risk of developing hormone refractory prostate or metastatic prostate cancer. Individual HRPCDETERMINANTS are include and are collectively referred to herein as, inter alia, “hormone refractory prostate cancer—associated proteins”, “HRPCDETERMINANT polypeptides”, or “HRPCDETERMINANT proteins”. The corresponding nucleic acids encoding the polypeptides are referred to as “hormone refractory prostate cancer—associated nucleic acids”, “hormone refractory prostate cancer—associated genes”, “HRPCDETERMINANT nucleic acids”, or “HRPCDETERMINANT genes”. Unless indicated otherwise, “HRPCDETERMINANT”, “hormone refractory prostate cancer—associated proteins”, “hormone refractory prostate cancer—associated nucleic acids” are meant to refer to any of the sequences disclosed herein. The corresponding metabolites of the HRPCDETERMINANT proteins or nucleic acids can also be measured, as well as any of the aforementioned traditional risk marker metabolites. HRPCDETERMINANTS include thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS).

Physiological markers of health status (e.g., such as age, family history, and other measurements commonly used as traditional risk factors) are referred to as “HRPCDETERMINANT physiology”. Calculated indices created from mathematically combining measurements of one or more, preferably one or moreof the aforementioned classes of HRPCDETERMINANTS are referred to as “HRPCDETERMINANT indices”.

“Clinical parameters” encompasses all non-sample or non-analyte biomarkers of subject health status or other characteristics, such as, without limitation, age (Age), ethnicity (RACE), gender (Sex), or family history (FamHX).

“Circulating endothelial cell” (“CEC”) is an endothelial cell from the inner wall of blood vessels which sheds into the bloodstream under certain circumstances, including inflammation, and contributes to the formation of new vasculature associated with cancer pathogenesis. CECs may be useful as a marker of tumor progression and/or response to antiangiogenic therapy.

“Circulating tumor cell” (“CTC”) is a tumor cell of epithelial origin which is shed from the primary tumor upon metastasis, and enters the circulation. The number of circulating tumor cells in peripheral blood is associated with prognosis in patients with metastatic cancer. These cells can be separated and quantified using immunologic methods that detect epithelial cells, and their expression of HRPCDETERMINANTS can be quantified by qRT-PCR, immunofluorescence, or other approaches.

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining HRPCDETERMINANTS and other HRPCDETERMINANTS are linear and non-linear equations and statistical classification analyses to determine the relationship between levels of HRPCDETERMINANTS detected in a subject sample and the subject's risk of metastatic disease. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a HRPCDETERMINANT selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcomes expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

“Negative predictive value” or “NPV” is calculated by TN/(TN+FN) or the true negative fraction of all negative test results. It also is inherently impacted by the prevalence of the disease and pre-test probability of the population intended to be tested.

See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

Finally, hazard ratios and absolute and relative risk ratios within subject cohorts defined by a test are a further measurement of clinical accuracy and utility. Multiple methods are frequently used to defining abnormal or disease values, including reference limits, discrimination limits, and risk thresholds.

“Analytical accuracy” refers to the reproducibility and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Positive predictive value” or “PPV” is calculated by TP/(TP+FP) or the true positive fraction of all positive test results. It is inherently impacted by the prevalence of the disease and pre-test probability of the population intended to be tested.

“Prostate cancer” is the malignant growth of abnormal cells in the prostate gland, capable of invading and destroying other prostate cells, and spreading (metastasizing) to other parts of the body, including bones and lymph nodes. As defined herein, the term “prostate cancer” includes Stage 1, Stage 2, Stage 3, and Stage 4 prostate cancer as determined by the Tumor/Nodes/Metastases (“TNM”) system which takes into account the size of the tumor, the number of involved lymph nodes, and the presence of any other metastases; or Stage A, Stage B, Stage C, and Stage D, as determined by the Jewitt-Whitmore system. ‘Hormone refractory prostate cancer” is prostate cancer that no longer responds to hormone therapy”

“Risk” in the context of the present invention, relates to the probability that an event will occur over a specific time period, as in the conversion to metastatic events, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a primary tumor to metastatic prostate cancer or to one at risk of developing a metastatic, or from at risk of a primary metastatic event to a more secondary metastatic event. Risk evaluation can also comprise prediction of future clinical parameters, traditional laboratory risk factor values, or other indices of cancer, either in absolute or relative terms in reference to a previously measured population. The methods of the present invention may be used to make continuous or categorical measurements of the risk of metastatic prostate cancer thus diagnosing and defining the risk spectrum of a category of subjects defined as being at risk for metastatic tumor. In the categorical scenario, the invention can be used to discriminate between normal and other subject cohorts at higher risk for metastatic tumors. Such differing use may require different HRPCDETERMINANT combinations and individualized panels, mathematical algorithms, and/or cut-off points, but be subject to the same aforementioned measurements of accuracy and performance for the respective intended use.

A “sample” in the context of the present invention is a biological sample isolated from a subject and can include, by way of example and not limitation, tissue biopsies, whole blood, serum, plasma, blood cells, endothelial cells, circulating tumor cells, lymphatic fluid, ascites fluid, interstitial fluid (also known as “extracellular fluid” and encompasses the fluid found in spaces between cells, including, inter alia, gingival cevicular fluid), bone marrow, cerebrospinal fluid (CSF), saliva, mucous, sputum, sweat, urine, or any other secretion, excretion, or other bodily fluids.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of tumor metastasis. A subject can be male or female. A subject can be one who has been previously diagnosed or identified as having primary tumor or a metastatic tumor, and optionally has already undergone, or is undergoing, a therapeutic intervention for the tumor. Alternatively, a subject can also be one who has not been previously diagnosed as having metastatic prostate cancer. For example, a subject can be one who exhibits one or more risk factors for metastatic prostate cancer.

“TN” is true negative, which for a disease state test means classifying a non-disease or normal subject correctly.

“TP” is true positive, which for a disease state test means correctly classifying a disease subject.

“Traditional laboratory risk factors” correspond to biomarkers isolated or derived from subject samples and which are currently evaluated in the clinical laboratory and used in traditional global risk assessment algorithms. Traditional laboratory risk factors for tumor metastasis include for example Gleason score, depth of invasion, vessel density, proliferative index, etc. Other traditional laboratory risk factors for tumor metastasis are known to those skilled in the art.

Method of Treating Prostate Cancer

Hormone refractory prostate cancer is treated, a symptom is alleviated, or the onset of androgen independent prostate cancer is delayed by administering to a subject a compound that inhibits the expression or activity of a serine threonine kinase. The serine threonine kinase is for example thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS).

By inhibiting the expression of a serine threonine kinase it is mean that the compound inhibits the expression of a serine threonine kinase nucleic acid (DNA or RNA) or a serine threonine kinase polyppetide. By inhibiting serine threonine kinase activity it is meant that the compound inhibits kinase activity i.e., phosphorylation or alternatively the compound inhibits activity independent of the phosphorylation activity of the serine threonine kinase polypeptide.

The subject has been diagnosed with hormone refractory prostate cancer. Alternatively, the subject has not has been diagnosed with hormone refractory prostate cancer.

Tissues or cells are directly contacted with an inhibitor. Alternatively, the inhibitor is administered systemically. Inhibitors are administered in an amount sufficient to decrease (e.g., inhibit) serine theronine kinase activity, i.e., phosphorylation; expression of a serine threonine kinase nucleic acid or polypeptide. For example, the serine threonine kinase inhibitor inhibits phosphorylation of AKT. Alternatively, the serine threonine kinase inhibitors are administered in an amount sufficient to decrease prostate cancer cell proliferation and or viability.

Serine threonine kinase inhibitors include for example peptides, peptidomimetics, small molecules, an antisense serine threonine kinase nucleic acid, a serine threonine kinase—specific short-interfering RNA, or a serine threonine kinase—specific ribozyme or other drugs

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules.

By the term “siRNA” is meant a double stranded RNA molecule which prevents translation of a target mRNA. Standard techniques of introducing siRNA into a cell are used, including those in which DNA is a template from which an siRNA RNA is transcribed. The siRNA includes a sense serine threonine kinase nucleic acid sequence, an anti-sense serine threonine kinase nucleic acid sequence or both. Optionally, the siRNA is constructed such that a single transcript has both the sense and complementary antisense sequences from the target gene, e.g., a hairpin.

Binding of the siRNA to an serine threonine kinase transcript in the target cell results in a reduction in serine threonine kinase production by the cell. The length of the oligonucleotide is at least 10 nucleotides and may be as long as the naturally-occurring serine threonine kinase transcript. Preferably, the oligonucleotide is 19-25 nucleotides in length. Most preferably, the oligonucleotide is less than 75, 50, 25 nucleotides in length.

Inhibitor of serine threonine kinase phoshorylation are known in the art. For example, various serien threonine kinase inhibitors can be found at Sigma Aldrich (St. Louis, Mo.). Serine threonine kinase inhibitors include for example is a thymidine kinase 1 (TK1) inhibitor, a uridine-cytidine kinase 2 (UCK2) inhibitor, a tyrosine kinase non-receptor 2 inhibitor (TNK2), a S-phase kinase-associated protein 2 (SKP2), a plasminogen activator, urokinase (PLAU) or a hepatocyte growth factor-regulated tyrosine kinase substrate (HGS)

The methods described herein lead to a reduction in the severity or the alleviation of one or more symptoms of a prostate cancer such as those described herein. Prostate cancer is diagnosed and or monitored, typically by a physician using standard methodologies Efficaciousness of treatment is determined in association with any known method for diagnosing or treating the prostate.

The compounds that inhibit serine theroine kinase expression or activity (also referred to herein as “active compounds”, and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

Methods and Uses of the Invention

The methods disclosed herein are used with subjects at risk for developing hormone refractory prostate cancer or metastatic prostate cancer, who may or may not have already been diagnosed with prostate cancer and subjects undergoing treatment and/or therapies for a primary tumor or metastatic prostate cancer. The methods of the present invention can also be used to monitor or select a treatment regimen for a subject who has a primary tumor or metastatic prostate cancer, and to screen subjects who have not been previously diagnosed as having metastatic prostate cancer, such as subjects who exhibit risk factors for hormone refractory prostate cancer or metastasis. Preferably, the methods of the present invention are used to identify and/or diagnose subjects who are asymptomatic for metastatic prostate cancer. “Asymptomatic” means not exhibiting the traditional signs and symptoms.

A subject having pr at risk of hormone refractory prostate cancer or metastatic prostate cancer scan be identified by measuring the amounts (including the presence or absence) of an effective number (which can be one or more) of HRPCDETERMINANTS in a subject-derived sample and the amounts are then compared to a reference value. Alterations in the amounts and patterns of expression of biomarkers, such as proteins, polypeptides, nucleic acids and polynucleotides, polymorphisms of proteins, polypeptides, nucleic acids, and polynucleotides, mutated proteins, polypeptides, nucleic acids, and polynucleotides, or alterations in the molecular quantities of metabolites or other analytes in the subject sample compared to the reference value are then identified.

A reference value can be relative to a number or value derived from population studies, including without limitation, such subjects having the same cancer, subject having the same or similar age range, subjects in the same or similar ethnic group, subjects having family histories of cancer, or relative to the starting sample of a subject undergoing treatment for a cancer. Such reference values can be derived from statistical analyses and/or risk prediction data of populations obtained from mathematical algorithms and computed indices of cancer metastasis. Reference HRPCDETERMINANT indices can also be constructed and used using algorithms and other methods of statistical and structural classification.

In one embodiment of the present invention, the reference value is the amount of HRPCDETERMINANTS in a control sample derived from one or more subjects who are not at risk or at low risk for developing hormone refractory prostate cancer or a metastatic tumor. In another embodiment of the present invention, the reference value is the amount of HRPCDETERMINANTS in a control sample derived from one or more subjects who are asymptomatic and/or lack traditional risk factors for hormone refractory prostate cancer or metastatic prostate cancer. In a further embodiment, such subjects are monitored and/or periodically retested for a diagnostically relevant period of time (“longitudinal studies”) following such test to verify continued absence of hormone refractory prostate cancer or metastatic prostate cancer (disease or event free survival). Such period of time may be one year, two years, two to five years, five years, five to ten years, ten years, or ten or more years from the initial testing date for determination of the reference value. Furthermore, retrospective measurement of HRPCDETERMINANTS in properly banked historical subject samples may be used in establishing these reference values, thus shortening the study time required.

A reference value can also comprise the amounts of HRPCDETERMINANTS derived from subjects who show an improvement in metastatic risk factors as a result of treatments and/or therapies for the cancer. A reference value can also comprise the amounts of HRPCDETERMINANTS derived from subjects who have confirmed disease by known invasive or non-invasive techniques, or are at high risk for developing hormone refractory prostate cancer or metastatic tumor, or who have suffered from hormone refractory prostate cancer or metastatic prostate cancer.

In another embodiment, the reference value is an index value or a baseline value. An index value or baseline value is a composite sample of an effective amount of HRPCDETERMINANTS from one or more subjects who do not have hormone refractory prostate cancer or a metastatic tumor, or subjects who are asymptomatic for hormone refractory prostate cancer or a metastatic prostate cancer. A baseline value can also comprise the amounts of HRPCDETERMINANTS in a sample derived from a subject who has shown an improvement in hormone refractory prostate cancer or metastatic tumor risk factors as a result of cancer treatments or therapies. In this embodiment, to make comparisons to the subject-derived sample, the amounts of HRPCDETERMINANTS are similarly calculated and compared to the index value. Optionally, subjects identified as having hormone refractory prostate cancer, a metastatic prostate tumor, or being at increased risk of developing metastatic prostate cancer are chosen to receive a therapeutic regimen to slow the progression the cancer, or decrease or prevent the risk of developing metastatic prostate cancer.

The progression of metastatic prostate cancer, or effectiveness of a cancer treatment regimen can be monitored by detecting a HRPCDETERMINANT in an effective amount (which may be one or more) of samples obtained from a subject over time and comparing the amount of HRPCDETERMINANTS detected. For example, a first sample can be obtained prior to the subject receiving treatment and one or more subsequent samples are taken after or during treatment of the subject. The cancer is considered to be progressive (or, alternatively, the treatment does not prevent progression) if the amount of HRPCDETERMINANT changes over time relative to the reference value, whereas the cancer is not progressive if the amount of HRPCDETERMINANTS remains constant over time (relative to the reference population, or “constant” as used herein). The term “constant” as used in the context of the present invention is construed to include changes over time with respect to the reference value.

For example, the methods of the invention can be used to discriminate the aggressiveness/and or accessing the stage of the tumor (e.g. Stage I, II, II or IV, hormone responsive or hormone refractory). This will allow patients to be stratified into high or low risk groups and treated accordingly.

Additionally, therapeutic or prophylactic agents suitable for administration to a particular subject can be identified by detecting a HRPCDETERMINANT in an effective amount (which may be one or more) in a sample obtained from a subject, exposing the subject-derived sample to a test compound that determines the amount (which may be one or more) of HRPCDETERMINANTS in the subject-derived sample. Accordingly, treatments or therapeutic regimens for use in subjects having a cancer, or subjects at risk for developing hormone refractory prostate cancer or metastatic tumor can be selected based on the amounts of HRPCDETERMINANTS in samples obtained from the subjects and compared to a reference value. One or more treatments or therapeutic regimens can be evaluated in parallel to determine which treatment or therapeutic regimen would be the most efficacious for use in a subject to delay onset, or slow progression of the cancer and or delay the onset of the development of hormone refractory prostate cancer

The present invention further provides a method for screening for changes in marker expression associated with hormone refractory prostate cancer or metastatic prostate cancer, by determining the amount (which may be one or more) of HRPCDETERMINANTS in a subject-derived sample, comparing the amounts of the HRPCDETERMINANTS in a reference sample, and identifying alterations in amounts in the subject sample compared to the reference sample.

The present invention further provides a method of treating a patient with a tumor, by identifying a patient with a tumor where an effective amount of HRPCDETERMINANTS are altered in a clinically significant manner as measured in a sample from the tumor, an treating the patient with a therapeutic regimen that prevents hormone refractory prostate cancer or prevents or reduces tumor metastasis.

Additionally the invention provides a method of selecting a tumor patient in need of adjuvant treatment by assessing the risk of metastasis in the patient by measuring an effective amount of HRPCDETERMINANTS where a clinically significant alteration one or more HRPCDETERMINANTS in a tumor sample from the patient indicates that the patient is in need of adjuvant treatment.

Information regarding a treatment decision for a tumor patient by obtaining information on an effective amount of HRPCDETERMINANTS in a tumor sample from the patient, and selecting a treatment regimen that prevents hormone refractory prostate cancer or prevents or reduces tumor metastasis in the patient if one or more HRPCDETERMINANTS are altered in a clinically significant manner.

If the reference sample, e.g., a control sample, is from a subject that does not have a hormone refractory prostate cancer or metastatic prostate cancer, or if the reference sample reflects a value that is relative to a person that has a high likelihood of rapid progression to hormone refractory prostate cancer or metastatic prostate cancer, a similarity in the amount of the HRPCDETERMINANT in the test sample and the reference sample indicates that the treatment is efficacious. However, a difference in the amount of the HRPCDETERMINANT in the test sample and the reference sample indicates a less favorable clinical outcome or prognosis.

By “efficacious”, it is meant that the treatment leads to a decrease in the amount or activity of a HRPCDETERMINANT protein, nucleic acid, polymorphism, metabolite, or other analyte. Assessment of the risk factors disclosed herein can be achieved using standard clinical protocols. Efficacy can be determined in association with any known method for diagnosing, identifying, or treating a metastatic disease.

The present invention also provides HRPCDETERMINANT panels including one or more HRPCDETERMINANTS that are indicative of a general physiological pathway associated with a metastatic lesion. For example, one or more HRPCDETERMINANTS that can be used to exclude or distinguish between different disease states or squeal associated with metastasis. A single HRPCDETERMINANT may have several of the aforementioned characteristics according to the present invention, and may alternatively be used in replacement of one or more other HRPCDETERMINANTS where appropriate for the given application of the invention.

The present invention also comprises a kit with a detection reagent that binds to one or more HRPCDETERMINANT proteins, nucleic acids, polymorphisms, metabolites, or other analytes. Also provided by the invention is an array of detection reagents, e.g., antibodies and/or oligonucleotides that can bind to one or more HRPCDETERMINANT proteins or nucleic acids, respectively. In one embodiment, the HRPCDETERMINANT are proteins and the array contains antibodies that bind one or more HRPCDETERMINANTS sufficient to measure a statistically significant alteration in HRPCDETERMINANT expression compared to a reference value. In another embodiment, the HRPCDETERMINANTS are nucleic acids and the array contains oligonucleotides or aptamers that bind an effective amount of HRPCDETERMINANTS sufficient to measure a statistically significant alteration in HRPCDETERMINANT expression compared to a reference value.

In another embodiment, the HRPCDETERMINANT are proteins and the array contains antibodies that bind an effective amount of HRPCDETERMINANTS sufficient to measure a statistically significant alteration in HRPCDETERMINANT expression compared to a reference value. In another embodiment, the HRPCDETERMINANTS are nucleic acids and the array contains oligonucleotides or aptamers that bind an effective amount of HRPCDETERMINANTS lsufficient to measure a statistically significant alteration in HRPCDETERMINANT expression compared to a reference value.

Also provided by the present invention is a method for treating one or more subjects at risk for developing hormone refractory prostate cancer or a metastatic tumor by detecting the presence of altered amounts of an effective amount of HRPCDETERMINANTS present in a sample from the one or more subjects; and treating the one or more subjects with one or more cancer-modulating drugs until altered amounts or activity of the HRPCDETERMINANTS return to a baseline value measured in one or more subjects at low risk for developing hormone refractory prostate canceror a metastatic disease, or alternatively, in subjects who do not exhibit any of the traditional risk factors for metastatic disease.

Also provided by the present invention is a method for treating one or more subjects having hormone refractory prostate cancer or metastatic tumor by detecting the presence of altered levels of an effective amount of HRPCDETERMINANTS present in a sample from the one or more subjects; and treating the one or more subjects with one or more cancer-modulating drugs until altered amounts or activity of the HRPCDETERMINANTS return to a baseline value measured in one or more subjects at low risk for developing hormone refractory prostate cancer or a metastatic tumor.

Also provided by the present invention is a method for evaluating changes in the risk of developing hormone refractory prostate cancer or metastatic prostate cancer in a subject diagnosed with cancer, by detecting an effective amount of HRPCDETERMINANTS (which may be one or more) in a first sample from the subject at a first period of time, detecting the amounts of the HRPCDETERMINANTS in a second sample from the subject at a second period of time, and comparing the amounts of the HRPCDETERMINANTS detected at the first and second periods of time.

Diagnostic and Prognostic Indications of the Invention

The invention allows the diagnosis and prognosis of a primary, locally invasive and/or metastatic prostate tumor or hormone refractory prostate cancer. The risk of developing hormone refractory prostate cancer or metastatic prostate cancer can be detected by measuring an effective amount of HRPCDETERMINANT proteins, nucleic acids, polymorphisms, metabolites, and other analytes (which may be one or more) in a test sample (e.g., a subject derived sample), and comparing the effective amounts to reference or index values, often utilizing mathematical algorithms or formula in order to combine information from results of multiple individual HRPCDETERMINANTS and from non-analyte clinical parameters into a single measurement or index. Subjects identified as having an increased risk of a metastatic prostate cancer or other metastatic cancer types can optionally be selected to receive treatment regimens, such as administration of prophylactic or therapeutic compounds to prevent or delay the onset of hormone refractory prostate cancer or metastatic prostate cancer.

The amount of the HRPCDETERMINANT protein, nucleic acid, polymorphism, metabolite, or other analyte can be measured in a test sample and compared to the “normal control level,” utilizing techniques such as reference limits, discrimination limits, or risk defining thresholds to define cutoff points and abnormal values. The “normal control level” means the level of one or more HRPCDETERMINANTS or combined HRPCDETERMINANT indices typically found in a subject not suffering from a metastatic tumor. Such normal control level and cutoff points may vary based on whether a HRPCDETERMINANT is used alone or in a formula combining with other HRPCDETERMINANTS into an index. Alternatively, the normal control level can be a database of HRPCDETERMINANT patterns from previously tested subjects who did not develop a metastatic tumor over a clinically relevant time horizon.

The present invention may be used to make continuous or categorical measurements of the risk of conversion to metastatic prostate cancer, or other metastatic cancer types thus diagnosing and defining the risk spectrum of a category of subjects defined as at risk for having a metastatic event. In the categorical scenario, the methods of the present invention can be used to discriminate between normal and disease subject cohorts. In other embodiments, the present invention may be used so as to discriminate those at risk for having hormone refractory prostate cancer or a metastatic event from those having more rapidly progressing (or alternatively those with a shorter probable time horizon to hormone refractory or a metastatic event) to hormone refractory prostate or a metastatic event from those more slowly progressing (or with a longer time horizon to a hormone refractory prostate or a metastatic event), or those having hormone refractory prostate cancer or metastatic cancer from normal. Such differing use may require different HRPCDETERMINANT combinations in individual panel, mathematical algorithm, and/or cut-off points, but be subject to the same aforementioned measurements of accuracy and other performance metrics relevant for the intended use.

Identifying the subject at risk of having hormone refractory prostate cancer or a metastatic event enables the selection and initiation of various therapeutic interventions or treatment regimens in order to delay, reduce or prevent that subject's conversion to hormone refractory prostate cancer or a metastatic disease state. Levels of an effective amount of HRPCDETERMINANT proteins, nucleic acids, polymorphisms, metabolites, or other analytes also allows for the course of treatment of a metastatic disease or metastatic event to be monitored. In this method, a biological sample can be provided from a subject undergoing treatment regimens, e.g., drug treatments, for cancer. If desired, biological samples are obtained from the subject at various time points before, during, or after treatment.

The present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progression to conditions like cancer or metastatic events, will be of value in the operations of, for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein.

A machine-readable storage medium can comprise a data storage material encoded with machine readable data or data arrays which, when using a machine programmed with instructions for using said data, is capable of use for a variety of purposes, such as, without limitation, subject information relating to metastatic disease risk factors over time or in response drug therapies. Measurements of effective amounts of the biomarkers of the invention and/or the resulting evaluation of risk from those biomarkers can implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system of the invention may also be considered to be implemented as a computer-readable storage medium, configured with a computer program, where the storage medium so configured causes a computer to operate in a specific and predefined manner to perform various functions described herein.

Levels of an effective amount of HRPCDETERMINANT proteins, nucleic acids, polymorphisms, metabolites, or other analytes can then be determined and compared to a reference value, e.g. a control subject or population whose metastatic state is known or an index value or baseline value. The reference sample or index value or baseline value may be taken or derived from one or more subjects who have been exposed to the treatment, or may be taken or derived from one or more subjects who are at low risk of developing hormone refractory prostate cancer or a metastatic event, or may be taken or derived from subjects who have shown improvements in as a result of exposure to treatment. Alternatively, the reference sample or index value or baseline value may be taken or derived from one or more subjects who have not been exposed to the treatment. For example, samples may be collected from subjects who have received initial treatment for cancer or a metastatic event and subsequent treatment for cancer or a metastatic event to monitor the progress of the treatment. A reference value can also comprise a value derived from risk prediction algorithms or computed indices from population studies such as those disclosed herein.

The HRPCDETERMINANTS of the present invention can thus be used to generate a “reference HRPCDETERMINANT profile” of those subjects who do not have cancer or are not at risk of having a metastatic event, and would not be expected to develop hormone refractory prostate cancer or a metastatic event. The HRPCDETERMINANTS disclosed herein can also be used to generate a “subject HRPCDETERMINANT profile” taken from subjects who have hormone refractory prostate cancer or are at risk for having a metastatic event. The subject HRPCDETERMINANT profiles can be compared to a reference HRPCDETERMINANT profile to diagnose or identify subjects at risk for developing hormone refractory prostate cancer or a metastatic event, to monitor the progression of disease, as well as the rate of progression of disease, and to monitor the effectiveness of treatment modalities. The reference and subject HRPCDETERMINANT profiles of the present invention can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

Differences in the genetic makeup of subjects can result in differences in their relative abilities to metabolize various drugs, which may modulate the symptoms or risk factors of cancer or metastatic events. Subjects that have cancer, or at risk for developing hormone refractory prostate cancer or a metastatic event can vary in age, ethnicity, and other parameters.

Accordingly, use of the HRPCDETERMINANTS disclosed herein, both alone and together in combination with known genetic factors for drug metabolism, allow for a pre-determined level of predictability that a putative therapeutic or prophylactic to be tested in a selected subject will be suitable for treating or preventing hormone refractory prostate cancer or a metastatic event in the subject.

To identify therapeutics or drugs that are appropriate for a specific subject, a test sample from the subject can also be exposed to a therapeutic agent or a drug, and the level of one or more of HRPCDETERMINANT proteins, nucleic acids, polymorphisms, metabolites or other analytes can be determined. The level of one or more HRPCDETERMINANTS can be compared to sample derived from the subject before and after treatment or exposure to a therapeutic agent or a drug, or can be compared to samples derived from one or more subjects who have shown improvements in risk factors (e.g., clinical parameters or traditional laboratory risk factors) as a result of such treatment or exposure.

A subject cell (i.e., a cell isolated from a subject) can be incubated in the presence of a candidate agent and the pattern of HRPCDETERMINANT expression in the test sample is measured and compared to a reference profile, e.g., a disease reference expression profile or a non-disease reference expression profile or an index value or baseline value. The test agent can be any compound or composition or combination thereof, including, dietary supplements. For example, the test agents are agents frequently used in cancer treatment regimens and are described herein.

The aforementioned methods of the invention can be used to evaluate or monitor the progression and/or improvement of subjects who have been diagnosed with a cancer, and who have undergone surgical interventions.

Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between subjects having cancer, or at risk for cancer or a metastatic event, is based on whether the subjects have, a “significant alteration” (e.g., clinically significant “diagnostically significant) in the levels of a HRPCDETERMINANT. By “effective amount” it is meant that the measurement of an appropriate number of HRPCDETERMINANTS (which may be one or more) to produce a “significant alteration,” (e.g. level of expression or activity of a HRPCDETERMINANT) that is different than the predetermined cut-off point (or threshold value) for that HRPCDETERMINANT(S) and therefore indicates that the subject has cancer or is at risk for having a metastatic event for which the HRPCDETERMINANT(S) is a determinant. The difference in the level of HRPCDETERMINANT between normal and abnormal is preferably statistically significant. As noted below, and without any limitation of the invention, achieving statistical significance, and thus the preferred analytical, diagnostic, and clinical accuracy, generally but not always requires that combinations of several HRPCDETERMINANTS be used together in panels and combined with mathematical algorithms in order to achieve a statistically significant HRPCDETERMINANT index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

By predetermined level of predictability it is meant that the method provides an acceptable level of clinical or diagnostic accuracy. Using such statistics, an “acceptable degree of diagnostic accuracy”, is herein defined as a test or assay (such as the test of the invention for determining the clinically significant presence of HRPCDETERMINANTS, which thereby indicates the presence of cancer and/or a risk of having a metastatic event) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods predict the presence or absence of a cancer, metastatic cancer or response to therapy with at least 75% accuracy, more preferably 80%, 85%, 90%, 95%, 97%, 98%, 99% or greater accuracy.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing cancer or metastatic event, and the bottom quartile comprising the group of subjects having the lowest relative risk for developing cancer or a metastatic event. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease; such is the case with total cholesterol and for many inflammatory biomarkers with respect to their prediction of future metastatic events. Often such lower diagnostic accuracy tests must be combined with additional parameters in order to derive meaningful clinical thresholds for therapeutic intervention, as is done with the aforementioned global risk assessment indices.

A health economic utility function is an yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category (such as those attic risk for having a metastatic event) has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the HRPCDETERMINANTS of the invention allows for one of skill in the art to use the HRPCDETERMINANTS to identify, diagnose, or prognose subjects with a pre-determined level of predictability and performance.

Construction of HRPCDETERMINANT Panels

Groupings of HRPCDETERMINANTS can be included in “panels.” A “panel” within the context of the present invention means a group of biomarkers (whether they are HRPCDETERMINANTS, clinical parameters, or traditional laboratory risk factors) that includes more than one HRPCDETERMINANT. A panel can also comprise additional biomarkers, e.g., clinical parameters, traditional laboratory risk factors, known to be present or associated with cancer or cancer metastasis, in combination with a selected group of the HRPCDETERMINANTS.

As noted above, many of the individual HRPCDETERMINANTS, clinical parameters, and traditional laboratory risk factors listed, when used alone and not as a member of a multi-biomarker panel of HRPCDETERMINANTS, have little or no clinical use in reliably distinguishing individual normal subjects, subjects at risk for having a metastatic event, and subjects having cancer from each other in a selected general population, and thus cannot reliably be used alone in classifying any subject between those three states. Even where there are statistically significant differences in their mean measurements in each of these populations, as commonly occurs in studies which are sufficiently powered, such biomarkers may remain limited in their applicability to an individual subject, and contribute little to diagnostic or prognostic predictions for that subject. A common measure of statistical significance is the p-value, which indicates the probability that an observation has arisen by chance alone; preferably, such p-values are 0.05 or less, representing a 5% or less chance that the observation of interest arose by chance. Such p-values depend significantly on the power of the study performed.

Despite this individual HRPCDETERMINANT performance, and the general performance of formulas combining only the traditional clinical parameters and few traditional laboratory risk factors, the present inventors have noted that certain specific combinations of one or more HRPCDETERMINANTS can also be used as multi-biomarker panels comprising combinations of HRPCDETERMINANTS that are known to be involved in one or more physiological or biological pathways, and that such information can be combined and made clinically useful through the use of various formulae, including statistical classification algorithms and others, combining and in many cases extending the performance characteristics of the combination beyond that of the individual HRPCDETERMINANTS. These specific combinations show an acceptable level of diagnostic accuracy, and, when sufficient information from multiple HRPCDETERMINANTS is combined in a trained formula, often reliably achieve a high level of diagnostic accuracy transportable from one population to another.

The general concept of how two less specific or lower performing HRPCDETERMINANTS are combined into novel and more useful combinations for the intended indications, is a key aspect of the invention. Multiple biomarkers can often yield better performance than the individual components when proper mathematical and clinical algorithms are used; this is often evident in both sensitivity and specificity, and results in a greater AUC. Secondly, there is often novel unperceived information in the existing biomarkers, as such was necessary in order to achieve through the new formula an improved level of sensitivity or specificity. This hidden information may hold true even for biomarkers which are generally regarded to have suboptimal clinical performance on their own. In fact, the suboptimal performance in terms of high false positive rates on a single biomarker measured alone may very well be an indicator that some important additional information is contained within the biomarker results—information which would not be elucidated absent the combination with a second biomarker and a mathematical formula.

Several statistical and modeling algorithms known in the art can be used to both assist in HRPCDETERMINANT selection choices and optimize the algorithms combining these choices. Statistical tools such as factor and cross-biomarker correlation/covariance analyses allow more rationale approaches to panel construction. Mathematical clustering and classification tree showing the Euclidean standardized distance between the HRPCDETERMINANTS can be advantageously used. Pathway informed seeding of such statistical classification techniques also may be employed, as may rational approaches based on the selection of individual HRPCDETERMINANTS based on their participation across in particular pathways or physiological functions.

Ultimately, formula such as statistical classification algorithms can be directly used to both select HRPCDETERMINANTS and to generate and train the optimal formula necessary to combine the results from multiple HRPCDETERMINANTS into a single index. Often, techniques such as forward (from zero potential explanatory parameters) and backwards selection (from all available potential explanatory parameters) are used, and information criteria, such as AIC or BIC, are used to quantify the tradeoff between the performance and diagnostic accuracy of the panel and the number of HRPCDETERMINANTS used. The position of the individual HRPCDETERMINANT on a forward or backwards selected panel can be closely related to its provision of incremental information content for the algorithm, so the order of contribution is highly dependent on the other constituent HRPCDETERMINANTS in the panel.

Construction of Clinical Algorithms

Any formula may be used to combine HRPCDETERMINANT results into indices useful in the practice of the invention. As indicated above, and without limitation, such indices may indicate, among the various other indications, the probability, likelihood, absolute or relative risk, time to or rate of conversion from one to another disease states, or make predictions of future biomarker measurements of metastatic disease. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from HRPCDETERMINANT results in the relevant training population. Amongst other uses, such formula may be intended to map the feature space derived from one or more HRPCDETERMINANT inputs to a set of subject classes (e.g. useful in predicting class membership of subjects as normal, at risk for having a metastatic event, having cancer), to derive an estimation of a probability function of risk using a Bayesian approach (e.g. the risk of cancer or a metastatic event), or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (R F, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual HRPCDETERMINANT measurement into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on Clinical Parameters such as age, gender, race, or sex, where specific formula are used solely on subjects within a class or continuously combining a Clinical Parameter as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al, (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al, 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula. An example of this is the presentation of absolute risk, and confidence intervals for that risk, derived using an actual clinical study, chosen with reference to the output of the recurrence score formula in the Oncotype Dx product of Genomic Health, Inc. (Redwood City, Calif.). A further modification is to adjust for smaller sub-populations of the study based on the output of the classifier or risk formula and defined and selected by their Clinical Parameters, such as age or sex.

Combination with Clinical Parameters and Traditional Laboratory Risk Factors

Any of the aforementioned Clinical Parameters may be used in the practice of the invention as a HRPCDETERMINANT input to a formula or as a pre-selection criteria defining a relevant population to be measured using a particular HRPCDETERMINANT panel and formula. As noted above, Clinical Parameters may also be useful in the biomarker normalization and pre-processing, or in HRPCDETERMINANT selection, panel construction, formula type selection and derivation, and formula result post-processing. A similar approach can be taken with the Traditional Laboratory Risk Factors, as either an input to a formula or as a pre-selection criterium.

Measurement of HRPCDETERMINANTS

The actual measurement of levels or amounts of the HRPCDETERMINANTS can be determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, amounts of HRPCDETERMINANTS can be measured using reverse-transcription-based PCR assays (RT-PCR), e.g., using primers specific for the differentially expressed sequence of genes or by branch-chain RNA amplification and detection methods by Panomics, Inc. Amounts of HRPCDETERMINANTS can also be determined at the protein level, e.g., by measuring the levels of peptides encoded by the gene products described herein, or subcellular localization or activities thereof using technological platform such as for example AQUA® (HistoRx, New Haven, Conn.) or U.S. Pat. No. 7,219,016. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed.

The HRPCDETERMINANT proteins, polypeptides, mutations, and polymorphisms thereof can be detected in any suitable manner, but is typically detected by contacting a sample from the subject with an antibody which binds the HRPCDETERMINANT protein, polypeptide, mutation, or polymorphism and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Immunoassays carried out in accordance with the present invention may be homogeneous assays or heterogeneous assays. In a homogeneous assay the immunological reaction usually involves the specific antibody (e.g., anti-HRPCDETERMINANT protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

Antibodies can also be useful for detecting post-translational modifications of HRPCDETERMINANT proteins, polypeptides, mutations, and polymorphisms, such as tyrosine phosphorylation, threonine phosphorylation, serine phosphorylation, glycosylation (e.g., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF) (Wirth, U. et al. (2002) Proteomics 2(10): 1445-51).

For HRPCDETERMINANT proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant K_{M} using known algorithms, such as the Hill plot, Michaelis-Menten equation, linear regression plots such as Lineweaver-Burk analysis, and Scatchard plot.

Using sequence information provided by the database entries for the HRPCDETERMINANT sequences, expression of the HRPCDETERMINANT sequences can be detected (if present) and measured using techniques well known to one of ordinary skill in the art. For example, sequences within the sequence database entries corresponding to HRPCDETERMINANT sequences, or within the sequences disclosed herein, can be used to construct probes for detecting HRPCDETERMINANT RNA sequences in, e.g., Northern blot hybridization analyses or methods which specifically, and, preferably, quantitatively amplify specific nucleic acid sequences. As another example, the sequences can be used to construct primers for specifically amplifying the HRPCDETERMINANT sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), e.g., using primers specific for the differentially expressed sequences. RNA can also be quantified using, for example, other target amplification methods (e.g., TMA, SDA, NASBA), or signal amplification methods (e.g., bDNA), and the like.

Alternatively, HRPCDETERMINANT protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. (See, WO 04/056456 and WO 04/088309, each of which are hereby incorporated by reference in their entireties) In this regard, other HRPCDETERMINANT analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others. Other HRPCDETERMINANT metabolites can be similarly detected using reagents that are specifically designed or tailored to detect such metabolites.

Kits

The invention also includes a HRPCDETERMINANT-detection reagent, e.g., nucleic acids that specifically identify one or more HRPCDETERMINANT nucleic acids by having homologous nucleic acid sequences, such as oligonucleotide sequences, complementary to a portion of the HRPCDETERMINANT nucleic acids or antibodies to proteins encoded by the HRPCDETERMINANT nucleic acids packaged together in the form of a kit. The oligonucleotides can be fragments of the HRPCDETERMINANT genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a Northern hybridization or a sandwich ELISA as known in the art.

For example, HRPCDETERMINANT detection reagents can be immobilized on a solid matrix such as a porous strip to form at least one HRPCDETERMINANT detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of HRPCDETERMINANTS present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by HRPCDETERMINANTS. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.).

Suitable sources for antibodies for the detection of HRPCDETERMINANTS include commercially available sources such as, for example, Abazyme, Abnova, Affinity Biologicals, AntibodyShop, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, nucleic acid probes, e.g., oligonucleotides, aptamers, siRNAs, antisense oligonucleotides, against any of the HRPCDETERMINANTS

### EXAMPLES

### Example 1

### General Method

Cell Lines and Culture Conditions

LHSR-AR {Berger, 2004 #15}, LNCaP {Horoszewicz, 1980 #23}, C4-2 {Wu, 1994 #18}, CL-1 {Patel, 2000 #16}, LNCaP-abl {Culig, 1999 #49} and LAPC4 {Klein, 1997 #34} cells have previously been described. LHSR-AR cells were cultured in PrEGM with growth supplements (Lonza). LNCaP were cultured in RPMI supplemented with 10% fetal bovine serum, 1 mM sodium pyruvate and 10 mM Hepes. C4-2, CL-1 and LNCaP-abl were cultured in phenol red free RMPI supplemented with 10% charcoal stripped fetal bovine serum, 1 mM sodium pyruvate and 10 mM Hepes. LAPC4 cells were cultured in IMDM supplemented with 10% fetal bovine serum and 1 nM R1881.

Myristoylated Kinase Library and Androgen Independence Screen

The myristoylated human kinase library, containing ORFs expressed from pWZL-Neo-Myr-Flag DEST retroviral vector, has been described. LHSR-AR cells were infected with the pooled library, consisting of 34 pools with 10-12 kinases per pool. Immunodeficient mice (Charles River, Boston, Mass.) were anesthetized with Avertin (Sigma) and castrated as described. 2×10⁶ cells resuspended in equal volumes of matrigel (Becton Dickinson) and PBS were subcutaneously implanted into castrated male mice or female mice, and tumor formation monitored.

Immunohistochemistry

Prostate cancer microarray slides (source) were immunostained with anti-TK1 antibody (1:50 dilution, Abcam, ab57757) using microwave-citrate antigen retrieval followed by standard IHC staining procedures. *Arrays were scored in a blinded manner by a pathologist on a scale of . . . * Paraffin embedded subcutaneous tumor sections were immunostained for AR as previously described using and anti-AR antibody (1:200 dilution, Santa Cruz, 441)

Immunoblotting

Immunoblots were performed as previously described {Boehm, 2005 #51}. The following antibodies were used: TK1 (Abcam, ab57757), AR (Santa Cruz, sc-7305), Flag (Sigma, M2), PDK1 (Becton Dickinson, 611070), p-PDK1 (Becton Dickinson, 558395), AKT (Cell Signaling, 9272), p-AKT1-Thr308 (Cell Signaling, 4056), p-AKT1-Ser473 (Cell Signaling, 9271), p-AKT1-Thr450 (Cell Signaling, 9267), p-AKT1-Tyr326 (Cell Signaling, 2968), Actin (Santa Cruz, sc-47778).

RNA Interference and Proliferation Assessment

Stable suppression of TK1 and AR were accomplished by using the pLKO.1-puro lentiviral shRNA constructs previously described {Moffat, 2006 #52}. The sequences targeted by the hairpins are as follows: shTK1-3, AGACCGTAATTGTGGCTGCAC (SEQ ID NO: 1); shTK1-4, GGGAAGCCGCCTATACCAAGA (SEQ ID NO: 2); shTK1-5, TGTCGGCTCTGCTACTTCAAG (SEQ ID NO: 3); shAR-1, CGCGACTACTACAACTTTCCA (SEQ ID NO: 4); shAR-4, CCTGCTAATCAAGTCACACAT (SEQ ID NO: 5); shAR-5, CCTTCAGACTTTGCTTCCCAT (SEQ ID NO: 6); shGFP, Experiments were performed in duplicate, in both the presence and absence of 1.5 ug/ml puromycin to monitor infection efficiency. Viable cells were counted using a Coulter Counter.

Invasion Assay

Matrigel invasion assay using matrigel biocoat invasion chambers (Becton Dickinson, 354480) was performed according to manufacturer instructions. Experiments were performed in triplicate.

Anchorage Independence Assay

Assay for growth of 3T3 cells in soft agar was conducted as previously described. Colonies were counted 3 weeks post plating using Image J software. Experiments were performed in triplicate.

Site Directed Mutagenesis

Site directed mutagenesis of TK1 and AKT1 were performed using the QuikChange II XL Site-Directed Mutagenesis kit (Stratagene). The primers used to generate the TK1E98A mutant were: E98A sense, TCATAGGCATCGACGCGGGGCAGTTTTTCCC (SEQ ID NO: 7); E98A antisense, GGGAAAAACTGCCCCGCGTCGATGCCTATGA (SEQ ID NO: 8). The primers used to generate the AKT1T308A mutant were: T308A sense, GGTGCCACCATGAAGGCCTTTTGCGGCACAC (SEQ ID NO: 9); T308A antisense, GTGTGCCGCAAAAGGCCTTCATGGTGGCACC (SEQ ID NO: 10). The primers used to generate the AKT1T308D mutant were: AKT1T308D sense, CGGTGCCACCATGAAGGACTTTTGCGGCACACCT (SEQ ID NO: 11); AKT1T308D antisense, AGGTGTGCCGCAAAAGTCCTTCATGGTGGCACCG (SEQ ID NO: 12).

Coimmunoprecipitation and Mass Spectrometer

Cells were lysed in 0.1% CHAPS buffer (0.1% CHAPS, 50 mM Tris-Hcl pH 7.4, 150 mM NaCl, 2 mM EDTA and protease inhibitor cocktail (Roche, Mannheim, Germany) as previously described {Arroyo, 2008 #61}. Lysates were incubated with FLAG M2 agarose beads (Sigma-Aldrich, St. Louis, Mo.) overnight at 4° C. Beads were washed in lysis buffer and eluted, and the eluate was concentrated as previously described. The immune complexes were separated on a 4-12% Bis-Tris NuPAGE gel (Invitrogen, Carlsbad, Calif.). The proteins were visualized by Colloidal Blue (Invitrogen, Carlsbad, Calif.) and were identified by mass spectrometry. For co-IP/Westerns, immune complexes were purified using anti-Flag (Sigma, F7425) and anti-AKT (Cell Signaling, 9272) antibodies.

### Example 2

### Identification of Kinases Responsible for Development of Hormone Resistant Prostate Cancer

In order to identify kinases whose expression is critical for the development of hormone resistant prostate cancer, we performed an in vivo forward genetic screen combining genetically defined androgen-dependent tumorigenic prostate cells with a library of human kinase open reading frames (ORFs) This library encodes >350 myristoylation-FLAG (MF) epitope tagged, and therefore potentially activated, human kinases and kinase related genes.

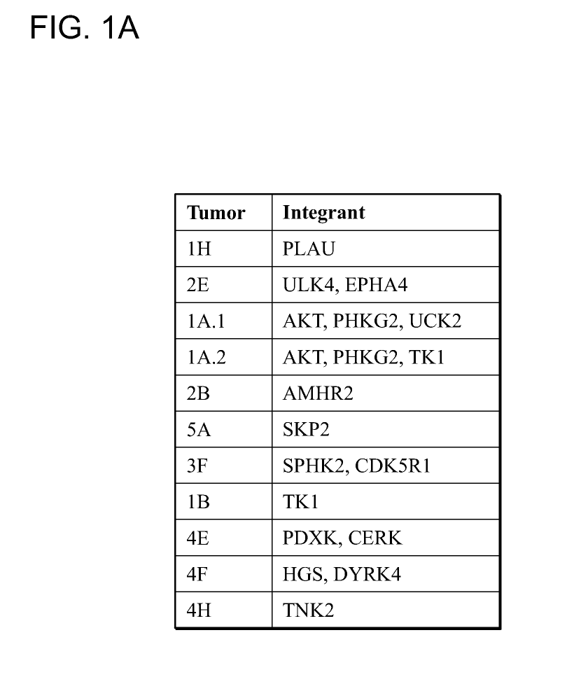

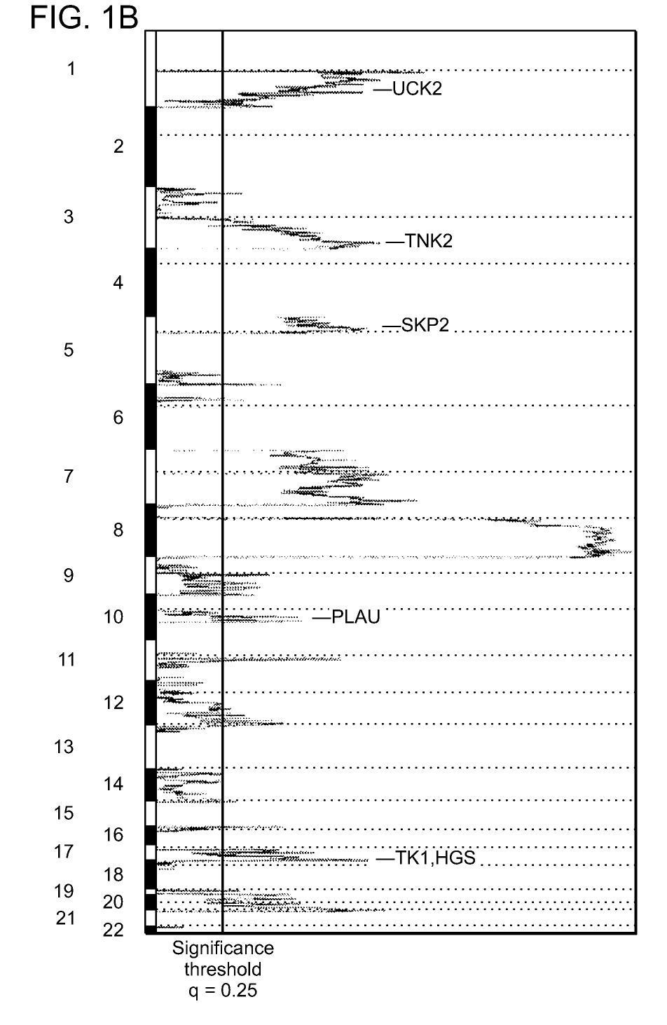

The MF kinase ORF library was introduced into LHSR-AR cells, dependent on androgens for tumorigenicity, by retroviral mediated gene transfer in pools of 12 kinases. This pool size was empirically determined to maximize the potential for finding activated kinases that induce the desired phenotype while minimizing the number of mice needed for these experiments (data not shown). Out of the total of 34 pools, 10 pools promoted androgen independent tumor formation in immunodeficient mice (data not shown). A total of 16 ORF integrants were identified in these tumors by PCR using vector specific primers (FIG. 1a).

Genes that promote cancer progression are often located in regions of chromosomal copy number gain. We explored whether any of the kinases that emerged from our screen are amplified in human prostate cancers. We used high-density single nucleotide polymorphism (SNP) arrays and GISTIC analysis to identify regions of chromosomal copy number alterations in 39 patient cancer specimens. 6 of the 16 kinases were found to be in regions of significant copy number gain (FIG. 1b).

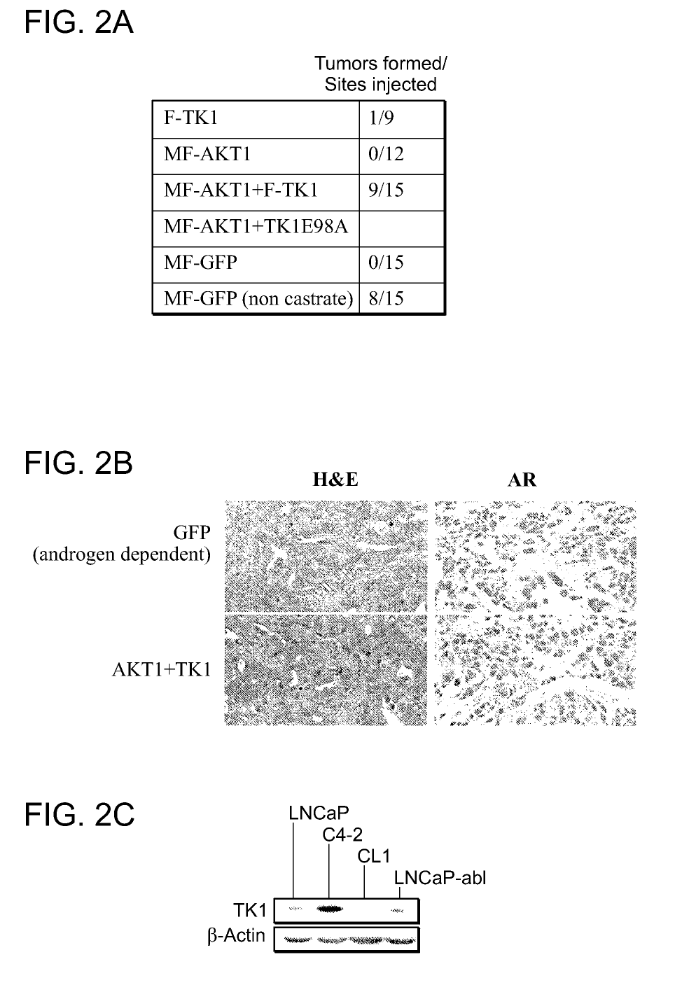

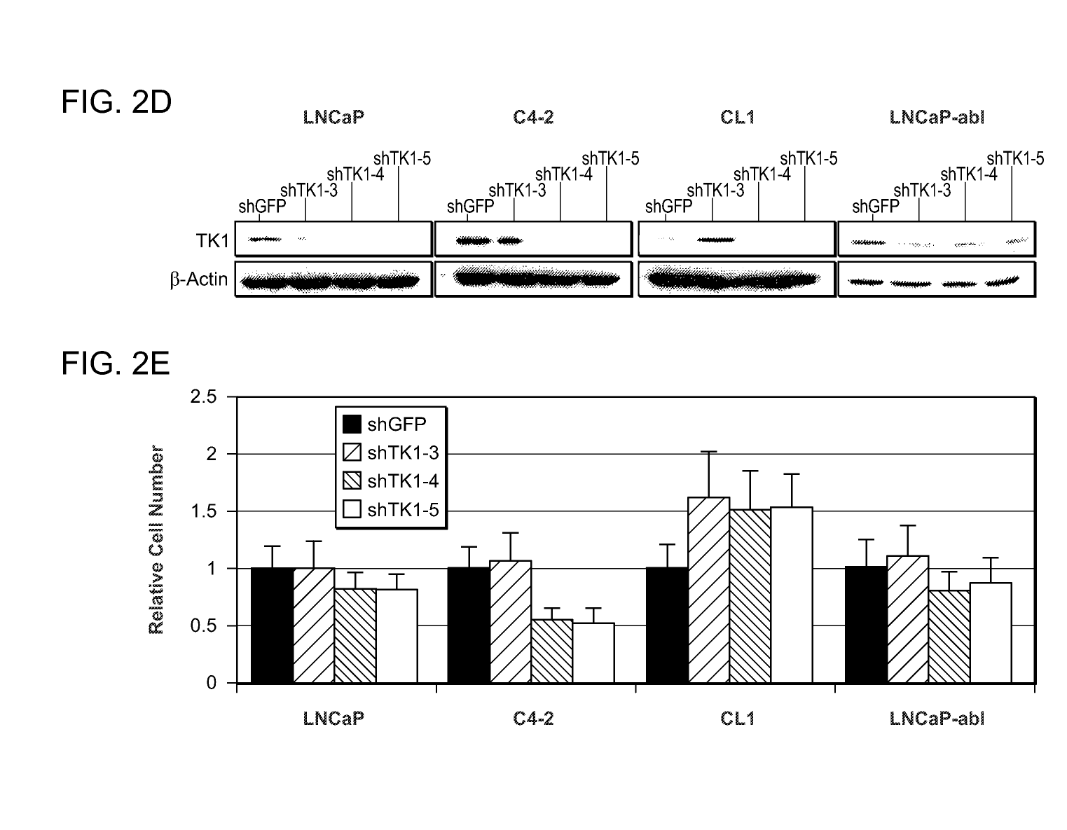

The ORF of one of these 6 kinases, thymidine kinase 1 (TK1), was identified in two tumors in our screen (FIG. 1a). In tumor 1B, it was the sole integrant identified, whereas in tumor 1A.1 it was identified along with AKT1 and PHKG2. Since AKT1 activation due to PTEN mutations or chromosomal copy deletions are commonly observed in hormone resistant prostate cancers, we hypothesized that while TK1 on its own may have the capacity to promote hormone resistance, it may synergize with AKT1 activation. To test this hypothesis, we injected castrated male mice with LHSR-AR cells infected with TK1 or AKT1 alone, or TK1 and AKT1 in combination. AKT1 on its own was unable to promote androgen independent tumor formation. While TK1 alone yielded one androgen independent tumor out of nine injections, the AKT1/TK1 combination induced androgen independent tumors at a rate greater than TK1 or AKT1 alone (FIG. 2a). Since TK1 is a cytosolic protein, we also tested nonmyristoylated TK1 cDNA in these experiments, and found the myristoylation tag was not necessary for the observed phenotype (data not shown). These findings indicate that TK1 and AKT1 synergize to promote androgen independent tumor growth.

### Example 3

### TK1 Activity Drives Androgen Dependent Tumor Formation

To test whether the kinase activity of TK1 is necessary for its capacity to drive androgen independent tumor formation, a kinase dead mutant of TK1 was generated by substituting catalytic glutamic acid at position +98 to alanine (E98A). This mutant construct was introduced into LHSR-AR/AKT cells, and the cells injected subcutaneously into castrated male mice. E98A-TK1 expressing cells were able to promote hormone independent tumor formation as efficiently as wild type TK1 expressing cells (FIG. 2a), suggesting that TK1 promotes hormone resistance independent of its kinase activity. E98A-TK1 expressing cells were unable to promote hormone independent tumor formation (FIG. 2a), suggesting that TK1 promotes hormone resistance through its kinase activity.

### Example 4

### TK1 Expression is More Prominent among Hormone Resistant Human Prostate Tumors

AR expression and signaling are retained in a large number of hormone refractory prostate tumors. In order to gain insight into whether TK1 and AKT1 promote the nuclear translocation of the androgen receptor (AR) in the absence of androgens, we conducted immunohistochemical AR staining of subcutaneous tumor sections. Nuclear staining for AR was detected in AKT1/TK1 tumors (FIG. 2b). PSA staining.

Since prostate cancer specimens display TK1 chromosomal copy number gain (FIG. 1b), with 52% of hormone refractory specimens analyzed displaying TK1 amplification compared to 12.5% of hormone dependent specimens (p-value 0.017) (FIG. 2c), we sought to determine whether TK1 protein is expressed more prominently in hormone refractory patient tumors. We conducted immunohistochemical TK1 staining of prostate tumor microarrays. Of the hormone sensitive and resistant tumors displaying TK1 expression, the percentage of TK1 positive cells in the tumors ranged from 1 to 15 percent, and the difference in mean percentage of TK1 positive cells in hormone sensitive versus resistant tumors was not significant (FIG. 2d, data not shown). We found, however, that while 64% of hormone resistant tumors displayed TK1 expression, only 27% of hormone resistant tumors were TK1 positive (p-value 0.033) (FIG. 2d), confirming that TK1 expression is more prominent among hormone resistant human prostate tumors.

### Example 5

### Prostate Cancer Cells Overexpressing TK1 are More Sensitive to TK1 Ablation

Next, we determined whether suppressing the expression of TK1 in prostate cancer cell lines that overexpress TK1 reverses their ability to proliferate or survive. Among a number of prostate cancer cell lines in which we assayed TK1 expression, the hormone resistant C4-2 {Wu, 1994 #18}, CL1 and LNCaP-abl lines, all derived from the hormone sensitive LNCaP line, expressed varying degrees of TK1 expression (FIG. 2e), with C4-2 cells expressing the highest and CL-1 the lowest TK1 levels. Suppression of TK1 expression using short hairpin RNA (shRNA) specific for TK1 (shTK1-4 and -5) reduced cell number 5 days post infections most significantly in C4-2 cultures, while it did not significantly affect CL-1 cells at this time (FIG. 2f) shTK1-3, which did not efficiently suppress TK1 expression, did not affect proliferation. It should be noted that by 12 days post infection, there were few cells remaining in all of the shTK1-4 and -5 cultures, indicating that while a basal level of TK1 expression may be necessary for in vitro proliferation, prostate cancer cells overexpressing TK1 are more sensitive to TK1 ablation.

### Example 6

### TK1 Overexpression does not Confer Significant Protection against Cell Death Induced by AR Knockdown

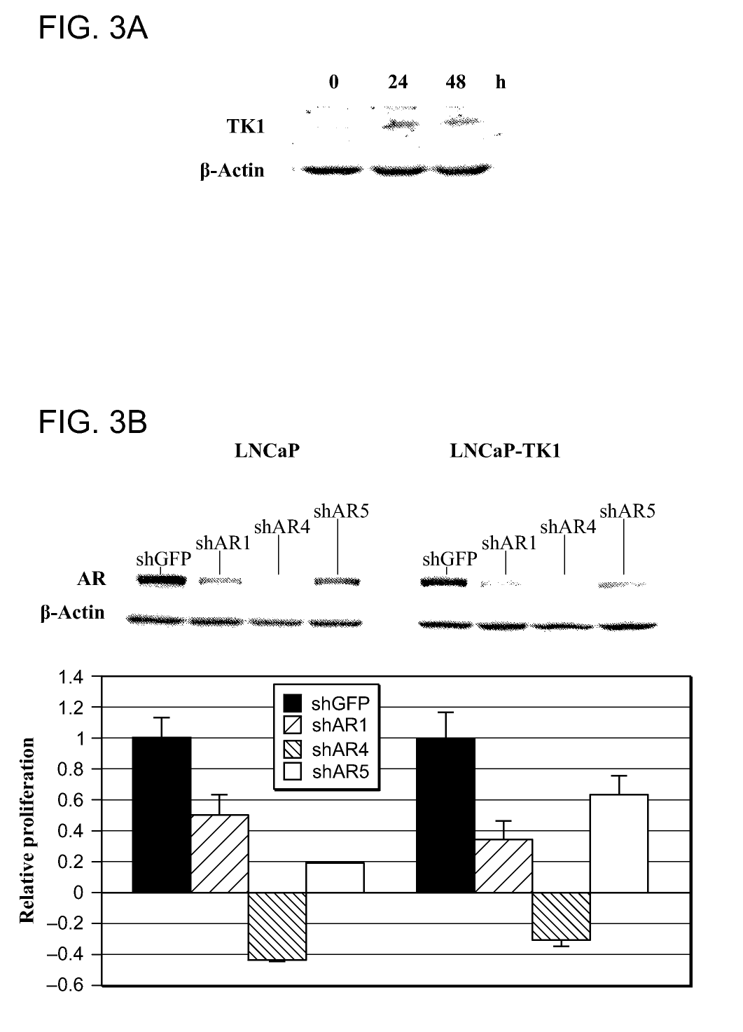

TK1 has been reported to be induced by androgens in the rat prostate. We determined whether TK1 expression is androgen induced in human prostate cells, and found that androgen treatment of androgen sensitive LNCaP cells resulted in increased TK1 protein expression (FIG. 3a). To elucidate whether TK1 is sufficient to replace AR signaling in promoting cell proliferation and survival, we silenced AR expression in LNCaP cells by RNAi in the presence or absence of ectopic TK1 expression, and monitored cell death. We found that TK1 overexpression did not confer significant protection against cell death induced by AR knockdown (FIG. 3b), suggesting that TK1 overexpression alone does not replace AR in promoting survival.

In order to elucidate the molecular pathways influenced by TK1, we sought to identify proteins that interact with TK1. TK1 immune complexes where purified by immunoprecipitation from androgen independent tumors that were derived by injecting LHSR-AR/TK1 cells into castrated mice. Interacting proteins were identified by mass spectrometry. Interestingly, among the proteins identified to interact with TK1 was AKT1 (4a).

### Example 7

### TK1 Influences AKT1 Phosphorylation

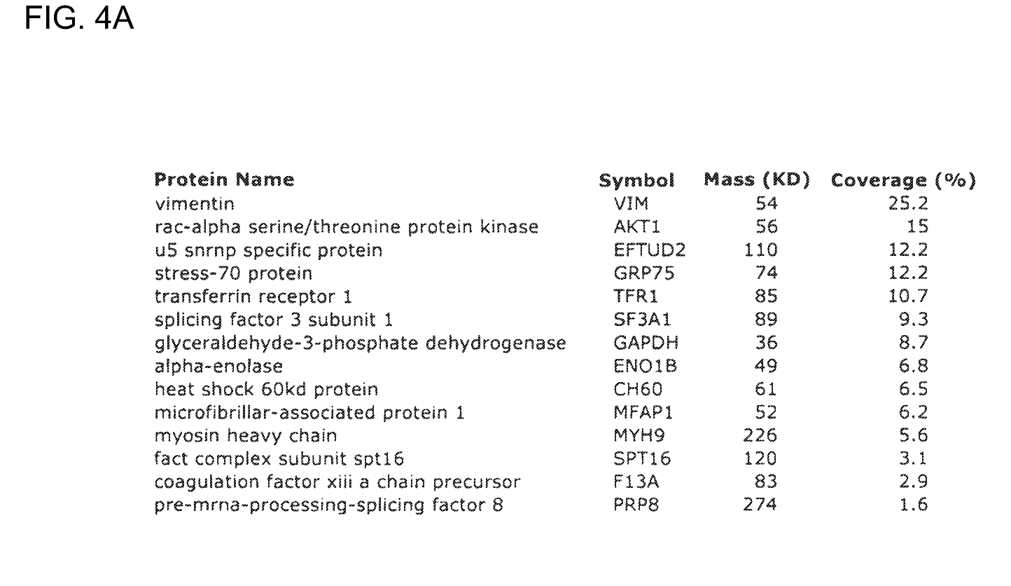

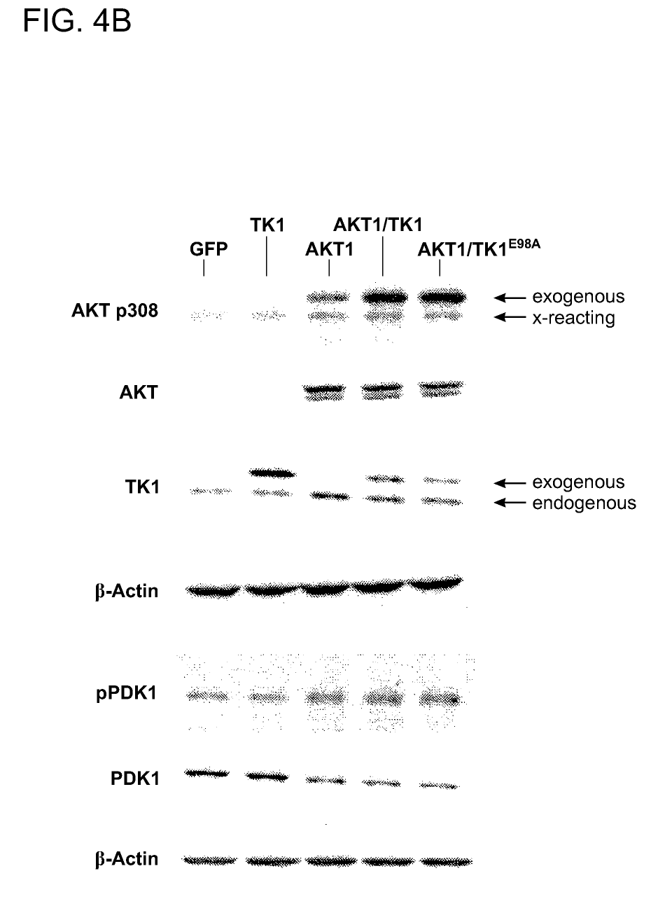

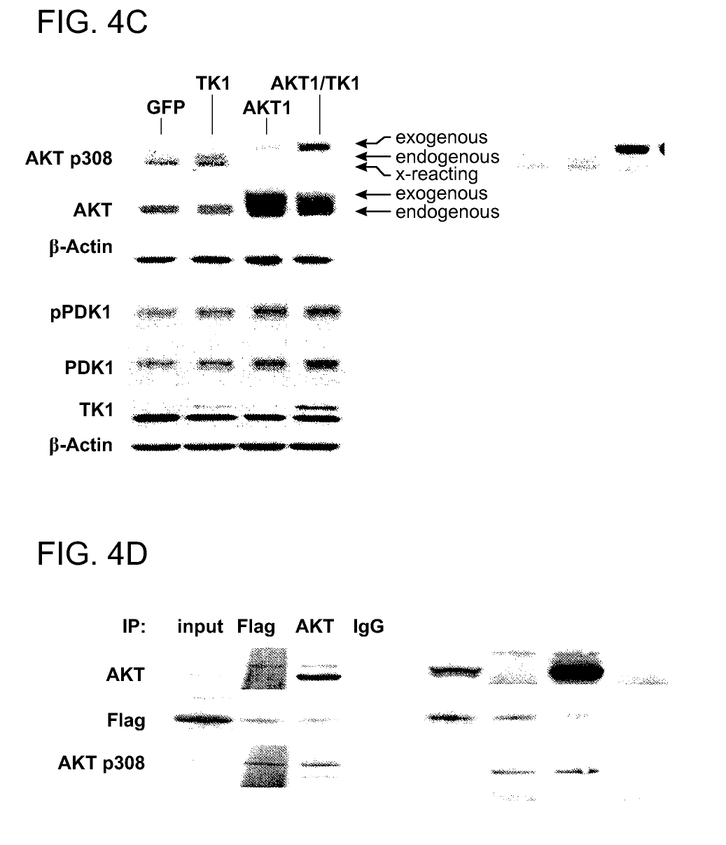

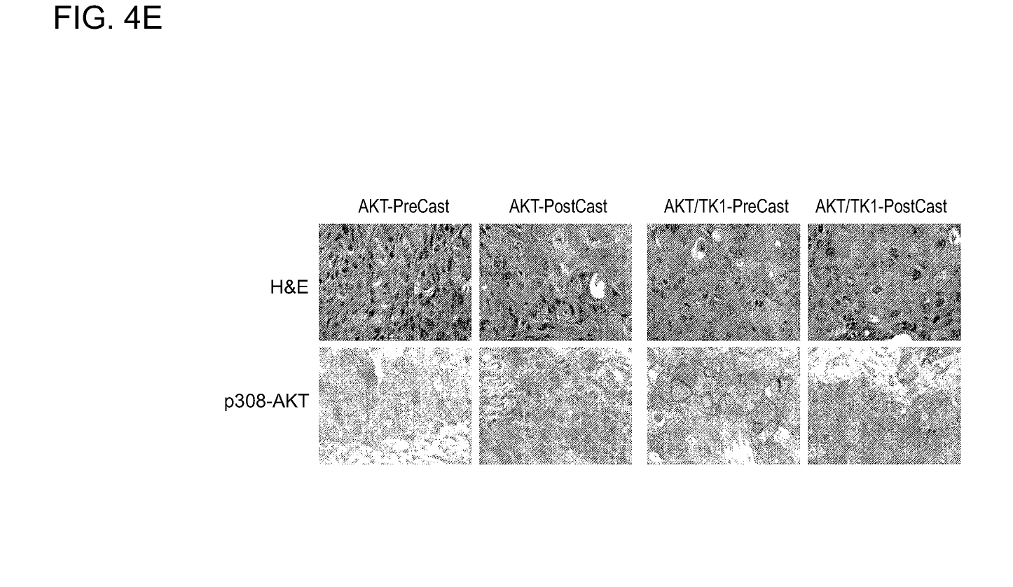

To test whether TK1 influences AKT1 phosphorylation, we assayed by immunoblotting the levels of p-AKT in LHSR-AR cells infected with TK1, AKT1, AKT1/TK1 and control GFP vectors. The combined expression of AKT1 and TK1 in LHSR-AR cells, compared to AKT1 alone, resulted in elevated pThr308-AKT levels. This increase was specific to the ectopic myristoylated AKT1 and was independent of TK1 kinase activity (FIG. 4b). Endogenous phospho-AKT was not detected. The levels of AKT phosphorylated at Ser473, Thr450 or Tyr326 were not affected (data not shown). TK1 expression in CL-1 prostate cells also resulted in elevated levels of both endogenous and exogenous pThr308-AKT (FIG. 4c). PDK1 is the kinase responsible for phosphorylating AKT at Thr308. TK1 expression in LHSR-AR or CL-1 cells did not promote a significant change in the expression levels of PDK1 or p-PDK1 (FIG. 4b,c), suggesting that TK1 may regulate p308-AKT1 independent of PDK1. The AKT1-TK1 interaction was confirmed by coimmunoprecipitation, where TK1 was found to interact with a form of AKT represented by a slower migrating band (FIG. 4d). Immunoblotting further demonstrated that this form of AKT is phosphorylated at Thr308 (FIG. 4d).

### Example 8

### TK1 Regulates p308-AKT In Vivo

Next, we sought to determine whether TK1 regulates p308-AKT in vivo. Noncastrate mice were injected subcutaneously with LHSR-AR/AKT and LHSR-AR/AKT/TK1 cells. Once tumors grew, they were harvested both pre and post castration, and tumor samples assayed for p308-AKT expression. p308-AKT expression was detected at the plasma membrane of cells in both AKT and AKT/TK1 precastrate tumors but staining intensity was significantly higher in AKT/TK1 tumors (FIG. 4e). Furthermore, while p308-AKT expression was substantially reduced following castration in AKT tumors, it was reduced to a lesser extent and still abundantly expressed in AKT/TK1 tumors (FIG. 4e). These data indicate that TK1 regulates p308-AKT in vivo.

### Example 9

### TK1 Ability to Enhance AKT1 Phosphorylation is Both Essential and Sufficient for Androgen Independence

To test whether the capacity of TK1 to enhance AKT1 phosphorylation is essential for its ability to synergize with AKT1 and promote hormone independence, we implanted LHSR-AR cells expressing TK1 and a mutant form of AKT1, where threonine in position 308 was substituted with alanine, in castrated nude mice (and failed to find hormone independent tumors?) (FIG. 4f). To determine whether the ability of TK1 to enhance AKT1 phosphorylation is sufficient to promote hormone independence, we implanted LHSR-AR cells expressing a mutant form of AKT1, where Threonine 308 was substituted with glutamate to mimic constitutive phosphorylation, into castrated nude mice. We also implanted cells expressing AKT1 in conjunction with PDK1 (FIG. 4f). Taken together, these indicate that while the ability of TK1 to enhance AKT1 phosphorylation is essential for androgen independence, it is not sufficient. In both cases, androgen independent tumor formation was observed (FIG. 4f). These indicate that the ability of TK1 to enhance AKT1 phosphorylation is both essential and sufficient for androgen independence.

### Example 10

### TK1 does not Enhance pAKT1 Stability

Finally, in order to determine whether TK1 promotes elevated levels of p-AKT1 by stabilizing p-AKT1 protein, p-AKT1 levels were monitored in CL1/AKT1 and CL1/AKT1/TK1 cells following cycloheximide treatment. The rate of p308-AKT1 degradation was slower in cells expressing TK1 (FIG. 4g), suggesting that TK1 enhances pAKT1 stability.* The rate of p308-AKT1 degradation was not affected by TK1 expression (FIG. 4g), suggesting that TK1 does not enhance pAKT1 stability.

We claim:

1. A method for treating or alleviating a symptom of hormone-refractory prostate cancer in a subject comprising administering to a subject in need thereof a therapeutically effective amount of a compound that inhibits the expression or activity of a serine threonine kinase.

2. A method of delaying the onset of androgen-independent prostate tumor growth in a subject comprising administering to a subject in need thereof a therapeutically effective amounts of a compound that inhibits the expression or activity of a serine threonine kinase.

3. The method of claim 1, wherein the compound inhibits the expression of a serine threonine kinase nucleic acid or polypeptide.

4. The method of claim 1, wherein the compound is a small molecule inhibitor, a small organic compound, a small inorganic compound, a nucleic acid, an antisense oligonucleotide, an siRNA, or an antibody.

5. The method of claim 1, wherein the compound inhibits the expression or activity of is a thymidine kinase 1 (TK1) a uridine-cytidine kinase 2 (UCK2), a tyrosine kinase non-receptor 2 (TNK2), a S-phase kinase-associated protein 2 (SKP2), a plasminogen activator, urokinase (PLAU) or a hepatocyte growth factor-regulated tyrosine kinase substrate (HGS).

6. The method of claim 1, wherein the compound inhibits a serine threonine kinase polypeptide activity independent of phosphorylation.

7. A method of assessing the risk of a subject developing a hormone-refractory prostate cancer comprising identifying an increase in expression or copy number of TK1 in a subject derived sample compared to a control sample wherein in said increase indicates an increased risk of developing hormone-refractory prostate cancer.

8. The method of claim 7, wherein the control sample is known normal tissue of the same tissue type as in the subject sample.

9. A method with a predetermined level of predictability for assessing a risk development of hormone-refractory prostate cancer or a metastatic prostate cancer in a subject comprising:
a. measuring the level of one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS) in a sample from the subject, and
b. measuring a clinically significant alteration in the level of the one or more kinases in the sample, wherein the alteration indicates an increased risk developing hormone-refractory prostate cancer or metastatic prostate cancer in the subject.

10. The method of claim 9, further comprising measuring at least one standard parameters associated with said cancer.

11. The method of claim 8, wherein said standard parameter is Gleason score or PSA.

12. The method of claim 9, wherein the level of said kinase is measured electrophoretically, immunochemically or by non-invasive imaging.

13. The method of claim 9, wherein the sample is a tumor biopsy, blood, or a circulating tumor cell in a biological fluid.

14. A method with a predetermined level of predictability for assessing a risk development of hormone-refractory prostate cancer or a metastatic prostate cancer in a subject comprising:
a. measuring the level of one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS) in a sample from the subject, and
b. comparing the level of the one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS) to a reference value.

15. The method of claim 14, wherein the reference value is an index value.

16. A method with a predetermined level of predictability for assessing the progression of a tumor in a subject comprising:
a. detecting the level of one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS) in a first sample from the subject at a first period of time;
b. detecting the level of one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS) in a second sample from the subject at a second period of time;
c. comparing the level of the one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS) in a sample from the subject detected in step (a) to the level detected in step (b), or to a reference value.

17. The method of claim 16, wherein the first sample is taken from the subject prior to being treated for the tumor.

18. The method of claim 16, wherein the second sample is taken from the subject after being treated for the tumor.

19. A method with a predetermined level of predictability for selecting a treatment regimen for a subject diagnosed with prostate cancer comprising:
a. detecting the level of one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS) in a first sample from the subject at a first period of time;
b. optionally detecting the level of one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS in a second sample from the subject at a second period of time;
c. comparing the level of one or more kinases selected from the group consisting of thymidine kinase 1 (TK1), uridine-cytidine kinase 2 (UCK2), a tyrosine kinase non-receptor 2 (TNK2), S-phase kinase-associated protein 2 (SKP2), plasminogen activator, urokinase (PLAU) and hepatocyte growth factor-regulated tyrosine kinase substrate (HGS) detected in step (a) to a reference value, or optionally, to the amount detected in step (b).

20. The method of claim 19, wherein the subject has previously been treated for the tumor.

21. The method of claim 19, wherein the first sample is taken from the subject prior to being treated for the tumor.

22. The method of claim 19, wherein the second sample is taken from the subject after being treated for the tumor.
